(19) United States
(12) Patent Application Publication
(10) Pub. No.: US 2012/0109020 A1
(43) Pub. Date: May 3, 2012

(54) METHODS FOR MODIFYING NEURAL TRANSMISSION PATTERNS

(75) Inventors: Timothy Andrew Wagner, Cambridge, MA (US); Uri Tzvi Eden, Somerville, MA (US)

(73) Assignee: HIGHLAND INSTRUMENTS, INC., Cambridge, MA (US)

(21) Appl. No.: 13/282,181

(22) Filed: Oct. 26, 2011

Related U.S. Application Data
(63) Continuation-in-part of application No. 11/764,468, filed on Jun. 18, 2007.
(60) Provisional application No. 60/814,843, filed on Jun. 19, 2006.

(51) Int. Cl. A61N 1/36; A61N 7/00; A61H 1/00; A61F 7/00; A61N 5/06

(52) U.S. Cl. 601/2; 607/2; 607/45; 607/96; 607/88; 601/1; 607/72

(57) ABSTRACT

The invention generally relates to methods for modifying neural transmission patterns. In certain embodiments, methods of the invention involve noninvasively applying a first type of energy to a region of neural tissue, noninvasively applying a second type of energy to the region of tissue, such that the combined effect modifies a neural transmission pattern between cells of the neural tissue.

### RELATED APPLICATIONS

The present application is a continuation-in-part of U.S. nonprovisional application Ser. No. 11/764,468, filed Jun. 18, 2007, which claims the benefit of and priority to U.S. provisional application Ser. No. 60/814,843, filed Jun. 19, 2006, the content of each of which is incorporated by reference herein in its entirety.

### FIELD OF THE INVENTION

The invention generally relates to methods for modifying neural transmission patterns.

### BACKGROUND

Neuromodulation is the control of nerve activity, and is usually implemented for the purpose of treating disease. Neuromodulation may be accomplished with surgical intervention, such as cutting an aberrant nerve tract. However, the semi-permanent nature of a surgical procedure leaves little room for later adjustment and optimization. Neuromodulation may also be accomplished with chemical agents or medications. Chemical agents or medications may be undesirable because, for example, many medications are difficult to deliver to specific anatomy, and because the titration (increasing or decreasing the dose of a medication) is a slow and imprecise way to achieve a desired effect on a specific target.

Neuromodulation may also be accomplished using energy-delivering devices. The stimulation may be applied invasively, e.g., by performing surgery to remove a portion of the skull and implanting electrodes in a specific location within brain tissue, or non-invasively, .e.g., transcranial direct current stimulation and transcranial magnetic stimulation.

The stimulation may act to modulate the plasticity of tissue (e.g., long term potentiation and/or long term depression). Long-term potentiation (LTP) involves the process of establishing an association between the firing of two cells or groups of cells. For instance, if an axon of cell A is near enough to excite a cell B and repeatedly or persistently takes part in firing cell B, an increase in the strength of the chemical synapse between the cells takes place such that A's efficiency, as one of the cells firing B, is increased. LTP has been shown to last from minutes to several months. Conditions for establishing LTP are favorable when a pre-synaptic neuron and a post-synaptic neuron are both depolarized in a synchronous manner. An opposite effect, long-term depression (LTD), has also been established. LTD is the weakening of a neuronal synapse that lasts from hours to months.

### SUMMARY

The invention generally relates to methods for modifying neural transmission patterns. Generally, the invention provides methods for modifying neural transmission patterns that involve noninvasively applying two different types of energy to a region of neural tissue such that the combined effect modifies a neural transmission pattern between cells of the neural tissue. The energy types may be applied sequentially or simultaneously. The modification may result in establishing a transmission patterns between the cells, strengthening a transmission patterns between the cells, weakening a transmission patterns between the cells, removing a transmission patterns between the cells, or a combination thereof. In certain embodiments, modifying transmission patterns results in long-term potentiation between the cells. Alternatively, modifying transmission patterns results in long-term depression between the cells.

Any combination of energy sources known in the art may be used with methods of the invention, e.g., optical, electromagnetic, electric, mechanical, or thermal. In certain embodiments, the energy is a combination of electrical energy and mechanical energy. The electrical energy may be an electric field. The electric filed may be pulsed, time varying, pulsed a plurality of time with each pulse being for a different length of time, or time invariant. The mechanical energy may be a mechanical field. The mechanical field may be pulsed, time varying, or pulsed a plurality of time with each pulse being for a different length of time. In certain embodiments, the electric field and/or the mechanical field is focused. In certain embodiments, the electric field is focused. In other embodiments, the mechanical field is focused. In still other embodiments, both the electrical and mechanical field are focused.

The energy may be applied to any region of tissue. In certain embodiments, the energy is applied to a structure or multiple structures within the brain or the nervous system such as the dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord. In particular embodiments, the tissue is neural tissue, and the affect of the stimulation alters neural function past the duration of stimulation.

Another aspect of the invention provides methods for modifying neural transmission patterns that involve noninvasively applying an electric field to a region of neural tissue, andnoninvasively applying a mechanical field to the region of neural tissue, such that the combined effect modifies a neural transmission pattern between cells of the neural tissue.

Another aspect of the invention provides methods for modifying neural transmission patterns that involve providing a noninvasive transcranial neural stimulator, and using the stimulator to modify a neural transmission pattern between cells of neural tissue.

### BRIEF DESCRIPTION OF THE DRAWINGS

The above-mentioned and other features and objects of this invention, and the manner of attaining them, will become more apparent and the invention itself will be better understood by reference to the following description of embodiments of the invention taken in conjunction with the accompanying drawings, wherein:

FIG. 1 is a plan view of one embodiment of an apparatus for stimulating biological tissue constructed in accordance with the principles of the present disclosure;

FIG. 2 is atop plan view of an exemplary embodiment of an apparatus for stimulating biological tissue constructed in accordance with the principles of the present disclosure;

FIG. 3 is atop plan view of an exemplary embodiment of an apparatus for stimulating biological tissue implementing a chemical source for altering permittivity constructed in accordance with the principles of the present disclosure;

FIG. 4 is atop plan view of an exemplary embodiment of an apparatus for stimulating biological tissue implementing a radiation source for altering permittivity constructed in accordance with the principles of the present disclosure; and

FIG. 5 is atop plan view of another exemplary embodiment of an apparatus for stimulating biological tissue implementing an optical beam for altering permittivity constructed in accordance with the principles of the present disclosure.

### DETAILED DESCRIPTION

Neuroplasticity refers to the ability of neural tissue (e.g., brain tissue or tissue of the nervous system) to change structurally and/or functionally as a result of input from the environment and/or via interactions between parts of the nervous system. Plasticity occurs on a variety of levels, ranging from cellular changes involved in learning, to large-scale changes involved in cortical remapping in response to injury. The most widely recognized forms of plasticity are learning, memory, and recovery from brain damage.

Plasticity can be categorized as short-term, lasting a few seconds or less, or long-term, which lasts from minutes to hours to days. Plasticity can also lead to permanent changes in the nervous system. Short-term synaptic enhancement results from an increase in the probability that synaptic terminals will release transmitters in response to pre-synaptic action potentials. Synapses will strengthen for a short time because of either an increase in size of the readily releasable pool of packaged neurotransmitter or an increase in the amount of packaged transmitter released in response to each action potential. Types of short term plasticity include synaptic augmentation, depression, facilitation, or neural facilitation, and post-tetanic potentiation. Synaptic augmentation is the increased efficacy of synapse lasting in the order of seconds. It has been found to be associated with increased efficiency with which action potentials cause release of vesicles containing transmitters. Depression is usually attributed to the depletion of the readily releasable vesicles. Depression can also arise from post-synaptic processes and from feedback activation of presynaptic receptor. Further short term alterations of the cell can also occur pre-synaptically and/or post-synaptically, which are elaborated on below.

Long-term depression and long-term potentiation are two forms of long-term plasticity, lasting minutes or more, that can occur at synapses. Long-term potentiation, commonly referred to as LTP, results in an increase in synaptic response, such as following potentiating pulses of electrical stimuli from the pre-synaptic neuron, that sustains at a level above the baseline response for hours or longer in the postsynaptic cell. LTP involves interactions between postsynaptic neurons and the specific presynaptic inputs that form a synaptic association, and is specific to the stimulated pathway of synaptic transmission. Long-term depression, commonly referred to as LDP, results in a decrease in synaptic strengths between cells and works in the opposite direction as LTP. Brief activation of an excitatory pathway can produce LTD of synaptic transmission in many areas of the brain. LTD can be induced by a minimum level of postsynaptic depolarization and simultaneous increase in the intracellular calcium concentration at the postsynaptic neuron. LTD can be initiated at inactive synapses if the calcium concentration is raised to the minimum required level by heterosynaptic activation, or if the extracellular concentration is raised. These alternative conditions capable of causing LTD depend on synaptic activity modifications.

The present invention is useful for allowing the practical application of a variety of LTP and LTD systems, and the invention has been found to be particularly suited for use in systems and methods dealing with generating LTP or LTD effects in neural circuits through the noninvasive application of two different types of energy to a region of neural tissue. Various embodiments of the present invention are directed toward the noninvasive application of two different types of energy to a region of neural tissue to produce LTP or LTD within the region of tissue. For LTP, the energy types can be used to concurrently stimulate a first and second portion of neurons in a synchronous manner. For LTD, the energy types can be used to stimulate a first and second portion of neurons in an asynchronous manner. Stimulation patterns can also be provided with single or multiple energy source types in any combination (e.g., providing ultrasound stimulation in conjunction with optical stimulation to another location, or ultrasound to one location and electrical stimulation to another location, or combined electromechanical stimulation to one location and optical stimulation to another location).

In certain embodiments, methods of the invention are used to stimulate two different areas of the brain. The stimulation of each area is coordinated in order to facilitate the development of either LTP or LTD between the two different areas of the brain. For example, each of the areas can be stimulated in a synchronous fashion to produce LTP. If the stimulation results in an increased rate of depolarization of the neurons, the probability that both areas of the brain will fire at the same time is likewise increased. Moreover, LTP may be developed where the stimulation results in one of the areas generating action potentials more readily in response to stimulus from the other area (e.g., by having a lower depolarization threshold). In order to produce LTD, the areas may be stimulated in an asynchronous fashion to produce an increased probability of the different areas firing independently from one another.

It is not required that two different portions of a neurons be stimulated (or two different regions of neural tissue or brain regions), as certain embodiments allow for the stimulation of one portion of a single neuron to bring about the plastic mechanisms (or a single tissue or brain site). For example, one could apply the appropriate stimulation energy patterns to a single cell to initiate (or suppress) LTP or LTD processes (this can also be done to augment the ongoing plastic processes)). As an example, one could provide tetanic stimulation patterns to a presynaptic neuron (such as via combined energies, where stimulation currents are generated through combined electrical and mechanical energies) to initiate potentiation of post-synaptic neuron (for examples of other plastic mechanisms see Principles of Neural Science by Eric Kandel, James Schwartz and Thomas Jessell (2000)).

One can provide stimulation with the appropriate combined frequency patterns of the two energy sources, to generate an inhibitory or facilitatory stimulation signal on a neural cell or tissue. Stimulation with two energy sources can be applied in a coordinated manner such as through methods described in Wagner (U.S. patent application Ser. No. 13/216,282). For example, an electrical current pulse pattern, generated from combined energies (such as through an electromechanical technique), can be generated in the tissue for stimulation of long term and/or short term plastic mechanisms (for example, electromechanical stimulation can be provided to generate 1 Hz current pulses and can be applied to a neuron, such as in the motor cortex to cause inhibitory effects, while a higher frequency stimulation can cause facilitatory effects). Any stimulation pattern known in the art of single energy stimulation sources can be rendered via the appropriate use of combined energy (where energy pattern effects between single energy sources and combined energy sources for stimulation can be matched, tuned, and/or compared using techniques described in Wagner (U.S. patent application Ser. No. 13/216,282). Such as for example, using pulses of electromechanical energy to generate currents in the tissues that mirror the effects of those seen in TMS, DBS, theta burst stimulation, vagal nerve stimulation, or any other forms of single energy source stimulation. The inhibitory/facilitatory effects could be also controlled as in described in Wagner (U.S. patent application Ser. No. 13/216,313).

Both long term and short term effects of stimulation for modulating neural transmission patterns can work pre-synaptically, through mechanisms in the synapse, and/or post-synaptically. Furthermore they can work on summed connection in networks of cells. Stimulation can be used to increase or decrease the strength of the connections between cells and/or nodes of a network. Stimulation with two combined energy sources can be used to bring about any known mechanism of plasticity (such as mechanisms described in Principles of Neural Science by Eric Kandel, James Schwartz and Thomas Jessell (2000)), and can be provided in inhibitory and/or facilitatory manners to enact and/or enforce desired plastic changes in the nervous system.

For example, one can modify the presynaptic cell to affect plastic mechanisms with stimulation by increasing or decreasing its transmembrane conductance to ions (such as Na+, K+, and/or Cl— where the first would be decreased and the latter two increased to inhibit a typical neuron, while facilitation would be caused by the opposite effects), affecting the presynaptic voltage gated channels to respond with different dynamics or to different input levels (such as decreasing the sensitivity of voltage gated Ca++ channels in the region of the presynaptic cleft of a typical presynaptic neuron so as to lower the Ca++ that can be used to affect the release of neurotransmitters at release sites requiring Ca++ such as for vesicle transmission) and/or to different voltage levels, and/or increasing/decreasing the amount and/or rate at which neurotransmitters may be released (and/or reabsorbed)). The presynaptic cell's impact on the plastic mechanisms can be affected by stimulation via altering any of the elements of the cell, such as for example the neural membrane, channels, neurotransmitter production, and/or cellular machinery necessary for releasing vesicles into the synapse. Furthermore, the presynaptic cell could be modified to respond to signals that are sent from the post synaptic cell that regulate synaptic transmission (such as for example affecting retrograde messengers, such as for example nitirc oxide transmission as is seen in some LTP systems and other neural transmission modulation mechanisms). By increasing or decreasing a neural cells output (in rate and/or magnitude) it can have the affect of facilitating or inhibiting postsynaptic cell with positive or negative connections, respectively (by reversing this, the opposite effect would occur).

One can further use stimulation to impact the transport and uptake of the neurotransmitter in the synapse between the pre and post-synaptic cells. For example, stimulation could be used to impact diffusion rates across the cleft (such as for example by impacting the ionic concentration in the extracellular medium via stimulation where particular stimulation patterns can be used to change sodium and potassium concentrations) or to alter the distance between the cells. Furthermore, stimulation can be used to alter the chemical processes used to breakdown neurotransmitters in the synaptic cleft, the mechanisms to remove waste products from the cleft, and/or impact their absorption in the post-synaptic cell (or the re-absorption of components broken down and/or remaining in the cleft that are needed by the presynaptic cell to generate neurotransmitter substances).

Furthermore, one can modify the post-synaptic cell with stimulation in ways to increase or decrease its affects on the plastic mechanisms. For example, stimulation can be used to increase or decrease its transmembrane conductance to ions, affecting the post-synaptic voltage gated channels to respond with different dynamics and/or to different voltage levels, and/or increasing/decreasing the sensitivity to incoming neurotransmitters (such as a modification of the individual neurotransmitter receptors to be more or less receptive to the transmitter, or altering the density/number of transmitters in the postsynaptic cell). This for example could be enacted via altering any of the elements of the cell, such as for example the neural membrane, channels, neurotransmitter production, and/or cellular machinery necessary for releasing vesicles into the synapse. Furthermore, the post-synaptic cell could be modified to release more or less retro-grade signals that are sent from the pre synaptic cell that regulate its function. The summed affect of all of the inputs (from cellular inputs, chemical inputs, and stimulation inputs) can determine whether cells function and/or outputs is increased or decreased.

The methods herein can be further integrated with other methods and/or devices that can be used to further enact synaptic changes, such as activity training, sensory stimulation, and/or biofeedback (i.e., while stimulation is being provided to increase the impact of these mechanisms). For instance one could provide facilitatory stimulation to presynaptic facilitatory neurons synchronously with a stimuli known to enact a positive firing of a post synaptic cell, such as for example during classic conditioning in an Aplysia (see Scientific American September, 1992). Furthermore, while a repetitive training task (such as manually constructing a item) is being conducted, stimulation can be provided simultaneously to the neural network responsible for control or memorization of the activity in order to enhance the effects of the training task (stimulation could be provided to strengthen neural connections necessary to perform the task). Furthermore, one could be undergoing a form of cognitive therapy, such as used for stroke recovery, and apply neurostimulation simultaneously to strengthen its effects (and/or during motor therapy). Furthermore, stimulation can be provided as a supplement to augment ongoing plastic mechanisms that are taking place in the brain independent of the brain stimulation methods, either through natural processes or via other interventions.

Any type of energy or combinations of energy known in the art may be used with methods of the invention. Exemplary types of energy sources include mechanical, optical, electromagnetic, thermal, or a combination thereof. In particular embodiments, the stimulation source is a mechanical field (i.e., acoustic field), such as that produced by an ultrasound device. In other embodiments, the stimulation source is an electrical field. In other embodiments, the stimulation source is a magnetic field. Other exemplary types of stimulation include Transcranial Direct Current Stimulation (TDCS), Transcranial Ultrasound (TUS)/Transcranial Doppler Ultrasound (TDUS), Transcranial Electrical Stimulation (TES), Transcranial Alternating Current Stimulation (TACS), Cranial Electrical Stimulation (CES), or Transcranial Magnetic Stimulation (TMS). Other exemplary types include implant methods such as deep brain stimulation (DBS), microstimulation, spinal cord stimulation (SCS), and vagal nerve stimulation (VNS). In other embodiments, the stimulation source may work in part through the alteration of the nervous tissue electromagnetic properties, where stimulation occurs from an electric source capable of generating an electric field across a region of tissue and a means for altering the impedance of tissue relative to the electric field, whereby the alteration of the tissue permittivity relative to the electric field generates a displacement current in the tissue (while conductivity can also be modified to affect the current). The means for altering the impedance may include a chemical source, optical source, mechanical source, thermal source, or electromagnetic source.

In certain embodiments, the type of energy is mechanical energy, such as that produced by an ultrasound device. In certain embodiments, the ultrasound device includes a focusing element so that the mechanical field may be focused. In other embodiments, the mechanical energy is combined with an additional type of energy, such as chemical, optical, electromagnetic, or thermal energy.

In other embodiments, the type of energy is electrical energy, such as that produced by placing at least one electrode in or near the tissue. In certain embodiments, the electrical energy is focused, and focusing may be accomplished based upon placement of electrodes. In other embodiments, the electrical energy is combined with an additional type of energy, such as mechanical, chemical, optical, electromagnetic, or thermal energy.

In particular embodiments, the energy is a combination of an electric field and a mechanical field. The electric field may be pulsed, time varying, pulsed a plurality of time with each pulse being for a different length of time, or time invariant. The mechanical filed may be pulsed, time varying, or pulsed a plurality of time with each pulse being for a different length of time. In certain embodiments, the electric field and/or the mechanical field is focused.

The exemplary embodiments of the apparatuses and methods disclosed can be employed in the area of neural stimulation, where amplified, focused, direction altered, and/or attenuated currents could be used to alter neural activity via directly stimulating neurons, depolarizing neurons, hyperpolarizing neurons, modifying neural membrane potentials, altering the level of neural cell excitability, and/or altering the likelihood of a neural cell firing. Likewise, the method for stimulating biological tissue may also be employed in the area of muscular stimulation, including cardiac stimulation, where amplified, focused, direction altered, and/or attenuated currents could be used to alter muscular activity via direct stimulation, depolarizing muscle cells, hyperpolarizing muscle cells, modifying membrane potentials, altering the level of muscle cell excitability, and/or altering the likelihood of cell firing. Similarly, it is envisioned that the present disclosure may be employed in the area of cellular metabolism, physical therapy, drug delivery, and gene therapy.

Detailed embodiments of the present disclosure are disclosed herein, however, it is to be understood that the described embodiments are merely exemplary of the disclosure, which may be embodied in various forms. Therefore, specific functional details disclosed herein are not to be interpreted as limiting, but merely as a basis for the claims and as a representative basis for teaching one skilled in the art to variously employ the present disclosure in virtually any appropriately detailed embodiment.

The components of the tissue stimulation method according to the present disclosure are fabricated from materials suitable for a variety medical applications, such as, for example, polymerics, gels, films, and/or metals, depending on the particular application and/or preference. Semi-rigid and rigid polymerics are contemplated for fabrication, as well as resilient materials, such as molded medical grade polyurethane, as well as flexible or malleable materials. The motors, gearing, electronics, power components, electrodes, and transducers of the method may be fabricated from those suitable for a variety of medical applications. The method according to the present disclosure may also include circuit boards, circuitry, processor components, etc. for computerized control. One skilled in the art, however, will realize that other materials and fabrication methods suitable for assembly and manufacture, in accordance with the present disclosure, also would be appropriate.

The following discussion includes a description of the components and exemplary methods for generating currents in biological tissues in accordance with the principles of the present disclosure. Alternate embodiments are also disclosed. Reference will now be made in detail to the exemplary embodiments of the present disclosure illustrated in the accompanying figures wherein like reference numerals indicate the similar parts throughout the figures.

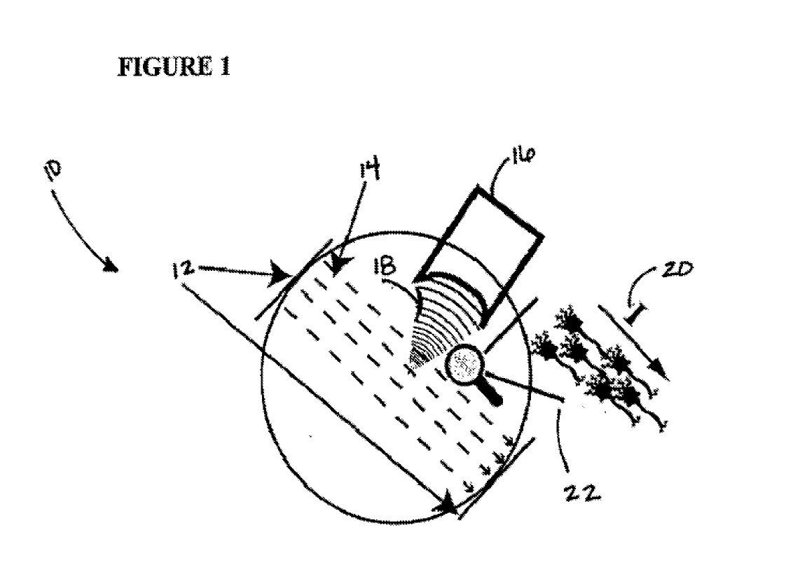

Turning now to FIG. 1, which illustrates an exemplary embodiment of an apparatus 10 to alter currents, e.g., amplify, focus, alter direction, and/or attenuate in the presence of an applied electric field or applied current source by the combined application of a mechanical field within a biological material to stimulate the biological cells and/or tissue in accordance with the present disclosure. For example, the apparatus 10 illustrated in FIG. 1 according to the present disclosure may be applied to the area of neural stimulation. An initial source electric field 14 results in a current in the tissue. The electric field 14 is created by an electric source, current or voltage source. As described in further detail below, the permittivity of the tissue is altered relative to the electric field, for example by a mechanical field, thereby generating an additional displacement current.

Electrodes 12 are applied to the scalp and generate a low magnitude electric field 14 over a large brain region. While electrodes 12 are used and applied to the scalp in this exemplary embodiment, it is envisioned that the electrodes may be applied to a number of different areas on the body including areas around the scalp. It is also envisioned that one electrode may be placed proximal to the tissue being stimulated and the other distant, such as one electrode on the scalp and one on the thorax. It is further envisioned that electric source could be mono-polar with just a single electrode, or multi-polar with multiple electrodes. Similarly, the electric source may be applied to tissue via any medically acceptable medium. It is also envisioned that means could be used where the electric source does not need to be in direct contact with the tissue, such as for example, inductive magnetic sources where the entire tissue region is placed within a large solenoid generating magnetic fields or near a coil generating magnetic fields, where the magnetic fields induce electric currents in the tissue.

The electric source may be direct current (DC) or alternating current (AC) and may be applied inside or outside the tissue of interest. Additionally, the source may be time varying. Similarly, the source may be pulsed and may be comprised of time varying pulse forms. The source may be an impulse. Also, the source according to the present disclosure may be intermittent.

A mechanical source such as an ultrasound source 16 is applied on the scalp and provides concentrated acoustic energy 18, i.e., mechanical field to a focused region of neural tissue, affecting a smaller number of neurons 22 than affected by the electric field 14, by the mechanical field 18 altering the tissue permittivity relative to the applied electric field 14, and thereby generating the altered current 20. The mechanical source may be any acoustic source such as an ultrasound device. Generally, such device may be a device composed of electromechanical transducers capable of converting an electrical signal to mechanical energy such as those containing piezoelectric materials, a device composed of electromechanical transducers capable of converting an electrical signal to mechanical energy such as those in an acoustic speaker that implement electromagnets, a device in which the mechanical source is coupled to a separate mechanical apparatus that drives the system, or any similar device capable of converting chemical, plasma, electrical, nuclear, or thermal energy to mechanical energy and generating a mechanical field.

Furthermore, the mechanical field could be generated via an ultrasound transducer that could be used for imaging tissue. The mechanical field may be coupled to tissue via a bridging medium, such as a container of saline to assist in the focusing or through gels and/or pastes which alter the acoustic impedance between the mechanical source and the tissue. The mechanical field may be time varying, pulsed, an impulse, or may be comprised of time varying pulse forms. It is envisioned that the mechanical source may be applied inside or outside of the tissue of interest. There are no limitations as to the frequencies that can be applied via the mechanical source, however, exemplary mechanical field frequencies range from the sub kHz to 1000 s of MHz. Additionally, multiple transducers providing multiple mechanical fields with similar or differing frequencies, and/or similar or different mechanical field waveforms may be used—such as in an array of sources like those used in focused ultrasound arrays. Similarly, multiple varied electric fields could also be applied. The combined fields, electric and mechanical, may be controlled intermittently to cause specific patterns of spiking activity or alterations in neural excitability. For example, the device may produce a periodic signal at a fixed frequency, or high frequency signals at a pulsed frequency to cause stimulation at pulse frequencies shown to be effective in treating numerous pathologies. Such stimulation waveforms may be those implemented in rapid or theta burst TMS treatments, deep brain stimulation treatments, epidural brain stimulation treatments, spinal cord stimulation treatments, or for peripheral electrical stimulation nerve treatments. The ultrasound source may be placed at any location relative to the electrode locations, i.e., within, on top of, below, or outside the same location as the electrodes as long as components of the electric field and mechanical field are in the same region. The locations of the sources should be relative to each other such that the fields intersect relative to the tissue and cells to be stimulated, or to direct the current alteration relative to the cellular components being stimulated.

The apparatus and method according to the present disclosure generates capacitive currents via permittivity alterations, which can be significant in magnitude, especially in the presence of low frequency applied electric fields. Tissue permittivities in biological tissues are much higher than most other non biological materials, especially for low frequency applied electric fields where the penetration depths of electric fields are highest. This is because the permittivity is inversely related to the frequency of the applied electric field, such that the tissue permittivity magnitude is higher with lower frequencies. For example, for electric field frequencies below 100,000 Hz, brain tissue has permittivity magnitudes as high as or greater than 10̂8 (100,000,000) times the permittivity of free space (8.854*10̂−12 farad per meter), and as such, minimal local perturbations of the relative magnitude can lead to significant displacement current generation. As the frequency of the electric field increases, the relative permittivity decreases by orders of magnitude, dropping to magnitudes of approximately 10̂3 times the permittivity of free space (8.854*10̂−12 farad per meter) for electric field frequencies of approximately 100,000 Hz. Additionally, by not being constrained to higher electric field frequencies, the method according to the present disclosure is an advantageous method for stimulating biological tissue due to lowered penetration depth limitations and thus lowered field strength requirements. Additionally, because displacement currents are generated in the area of the permittivity change, focusing can be accomplished via the ultrasound alone. For example, to generate capacitive currents via a permittivity perturbation relative to an applied electric field as described above, broad DC or a low frequency electric source field well below the cellular stimulation threshold is applied to a brain region but stimulation effects are locally focused in a smaller region by altering the tissue permittivity in the focused region of a mechanical field generated by a mechanical source such as an ultrasound source. This could be done noninvasively with the electrodes and the ultrasound device both placed on the scalp surface such that the fields penetrate the tissue surrounding the brain region and intersect in the targeted brain location, or with one or both of the electrodes and/or the ultrasound device implanted below the scalp surface (in the brain or any of the surrounding tissue) such that the fields intersect in the targeted region.

A displacement current is generated by the modification of the permittivity in the presence of the sub threshold electric field and provides a stimulatory signal. In addition to the main permittivity change that occurs in the tissues, which is responsible for stimulation (i.e., the generation of the altered currents for stimulation), a conductivity change could also occur in the tissue, which secondarily alters the ohmic component of the currents. In a further embodiment, the displacement current generation and altered ohmic current components may combine for stimulation. Generally, tissue conductivities vary slightly as a function of the applied electric field frequency over the DC to 100,000 Hz frequency range, but not to the same degree as the permittivities, and increase with the increasing frequency of the applied electric field. Additionally in biological tissues, unlike other materials, the conductivity and permittivity do not show a simple one-to-one relationship as a function of the applied electric field frequency. The permittivity ranges are as discussed above.

Although the process described may be accomplished at any frequency of the applied electric field, the method in an exemplary embodiment is applied with lower frequency applied electric fields due to the fact the permittivity magnitudes of tissues, as high as or greater than 10̂8 times the permittivity of free space, and the electric field penetration depths are highest for low frequency applied electric fields. Higher frequency applied electric fields may be less desirable as they will require greater radiation power to penetrate the tissue and/or a more pronounced mechanical source for permittivity alteration to achieve the same relative tissue permittivity change, i.e., at higher applied electric field frequencies the permittivity of the tissue is lower and as such would need a greater overall perturbation to have the same overall change in permittivity of a tissue as at a lower frequency. Applied electric field frequencies in the range of DC to approximately 100,000 Hz frequencies are advantageous due to the high tissue permittivity in this frequency band and the high penetration depth for biological tissues at these frequencies. In this band, tissues are within the so called ‘alpha dispersion band where relative tissue permittivity magnitudes are maximally elevated (i.e., as high as or greater than 10̂8 times the permittivity of free space). Frequencies above approximately 100,000 to 1,000,000 Hz for the applied electric fields are still applicable for the method described in generating displacement currents for the stimulation of biologic cells and tissue, however, both the tissue permittivity and penetration depth are limited for biological tissues in this band compared to the previous band but displacement currents of sufficient magnitude can still be generated for some applications. In this range, the magnitude of the applied electric field will likely need to be increased, or the method used to alter the permittivity relative to the applied electric field increased to bring about a greater permittivity change, relative to the tissue's permittivity magnitude for the applied electric field frequency. Additionally, due to potential safety concerns for some applications, it may be necessary to limit the time of application of the fields or to pulse the fields, as opposed to the continuous application that is possible in the prior band. For tissues or applications where the safety concerns preclude the technique in deeper tissues, the technique could still be applied in more superficial applications in a noninvasive manner or via an invasive method. Higher frequency applied electric fields, above 1,000,000 to 100,000,000 Hz, could be used in generating displacement currents for the stimulation of biologic cells and tissue. However, this would require a more sufficient permittivity alteration or electromagnetic radiation, and as such is less than ideal in terms of safety than the earlier bands. For frequencies of the applied electric field above 100,000,000 Hz, biologic cell and tissue stimulation may still be possible, but may be limited for specialized applications that require less significant displacement currents.

The focus of the electric and mechanical fields to generate an altered current according to the present disclosure may be directed to various structures within the brain or nervous system including but not limited to dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe,sub-cortical structures, spinal cord, nerve roots, sensory organs, and peripheral nerves.

The focused tissue may be selected such that a wide variety of pathologies may be treated. Such pathologies that may be treated include but are not limited to Multiple Sclerosis, Amyotrophic Lateral Sclerosis, Alzheimer's Disease, Dystonia, Tics, Spinal Cord Injury, Traumatic Brain Injury, Drug Craving, Food Craving, Alcohol Craving, Nicotine Craving, Stuttering, Tinnitus, Spasticity, Parkinson's Disease, Parkinsonianism, Obsessions, Depression, Schizophrenia, Bipolar Disorder, Acute Mania, Catonia, Post-Traumatic Stress Disorder, Autism, Chronic Pain Syndrome, Phantom Limb Pain, Epilepsy, Stroke, Auditory Hallucinations, Movement Disorders, Neurodegenerative Disorders, Pain Disorders, Metabolic Disorders, Addictive Disorders, Psychiatric Disorders, Traumatic Nerve Injury, and Sensory Disorders. Furthermore, electric and mechanical fields to generate an altered current may be focused on specific brain or neural structures to enact procedures including sensory augmentation, sensory alteration, anesthesia induction and maintenance, brain mapping, epileptic mapping, neural atrophy reduction, neuroprosthetic interaction or control with nervous system, stroke and traumatic injury neurorehabilitation, bladder control, assisting breathing, cardiac pacing, muscle stimulation, and treatment of pain syndromes, such as those caused by migraine, neuropathies, and low-back pain; or internal visceral diseases, such as chronic pancreatitis or cancer. The methods herein could be expanded to any form of arthritis, impingement disorders, overuse injuries, entrapment disorders, and/or any muscle, skeletal, or connective tissue disorder which leads to chronic pain, central sensitization of the pain signals, and/or an inflammatory response.

In the focused region of tissue to which the mechanical fields and electrical fields are delivered, the excitability of individual neurons can be heightened to the point that the neurons can be stimulated by the combined fields, or be affected such as to cause or amplify the alteration of the neural excitability caused by the altered currents, either through an increase or decrease in the excitability of the neurons. This alteration of neural excitability can last past the duration of stimulation and thus be used as a basis to provide lasting treatment. Additionally, the combined fields can be provided in multiple, but separate sessions to have a summed, or carry-over effect, on the excitability of the cells and tissue. The combined fields can be provided prior to another form of stimulation, to prime the tissue making it more or less susceptible to alternate, follow-up forms of stimulation. Furthermore, the combined fields can be provided after an alternate form of stimulation, where the alternate form of stimulation is used to prime the tissue to make it more or less susceptible to the form of stimulation disclosed herein. Furthermore, the combined fields could be applied for a chronic period of time.

The electrical and/or mechanical fields may be provided in focused and/or non-focused form, where focusing may be enacted through methods described in Wagner (U.S. patent application Ser. No. 13/169,288) where either element can be focused relative to a broader field of the other type.

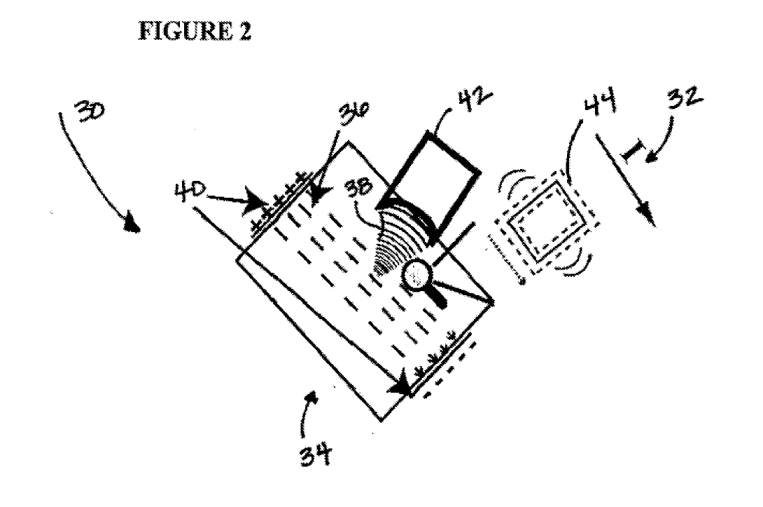

FIG. 2 illustrates a set up 30 to perform a method for generating an altered current with a newly generated displacement current 32 for stimulation in biologic tissue 34 through the combined effects of an electric field 36 and a mechanical field 38. A tissue or composite of tissues 34 is placed adjacent to the anode and cathode of an electric source 40 which generates an electric field 36. The electric field 36 is combined with a mechanical, e.g., ultrasound field 38 which can be focused on the tissue 34 and generated via an ultrasound transducer 42. In a sub-region of tissue 44 where the mechanical field 38 is focused and intersects with the electric field 36, a displacement current 32 is generated. By vibrating and/or mechanically perturbing the sub-region of tissue 44, the permittivity of the tissue 44 can be altered relative to the applied electric field 36 to generate a displacement current 32 in addition to the current that would be present due to the source electric field 36 and altered due to conductivity changes in the tissue caused by the mechanical perturbation.

By providing the mechanical field 38 to the sub region of tissue 44, the permittivity can be altered within the electric field 36 by either new elements of the sub region of tissue 44 vibrating in and out of the electric field such that the continuum permittivity of the tissue is changed relative to the electric field 36, or that the bulk properties of the sub region of tissue 44 and the permittivity, or tissue capacitance, change due to the mechanical perturbation. An example of altering the permittivity within the electric field can occur when a cell membrane and extra-cellular fluid, both of different permittivities, are altered in position relative to the electric field by the mechanical field. This movement of tissues of different permittivity relative to the electric field will generate a new displacement current. The tissues could have permittivity values as high as or greater than 10̂8 times the permittivity of free space, differ by orders of magnitude, and/or have anisotropic properties such that the tissue itself demonstrates a different permittivity magnitude depending on the relative direction of the applied electric field. An example of altering permittivity of the bulk tissue occurs where the relative permittivity constant of the bulk tissue is directly altered by mechanical perturbation in the presence of an electric field. The mechanical source, i.e., ultrasound source may be placed at any location relative to the electrode locations, i.e., within or outside the same location as the electrodes, as long as components of the electric field and mechanical field are in the same region.

Tissue permittivities can be altered relative to the applied electric fields via a number of methods. Mechanical techniques can be used to either alter the bulk tissue permittivity relative to an applied electric field or move tissue components of differing permittivities relative to an applied electric field. There are no specific limitations to the frequency of the mechanical field that is applied as previously discussed, however, exemplary frequencies range from the sub kHz to 1000 s of MHz. A second electromagnetic field could be applied to the tissue, at a different frequency than the initial frequency of the applied electromagnetic field, such that it alters the tissue permittivity at the frequency dependent point of the initially applied electric field. An optical signal could also be focused on the tissues to alter the permittivity of the tissue relative to an applied electric field. A chemical agent or thermal field could also be applied to the tissues to alter the permittivity of the tissue relative to an applied electric field. These methods could also be used in combination to alter the tissue permittivity relative to an applied electric field via invasive or noninvasive methods.

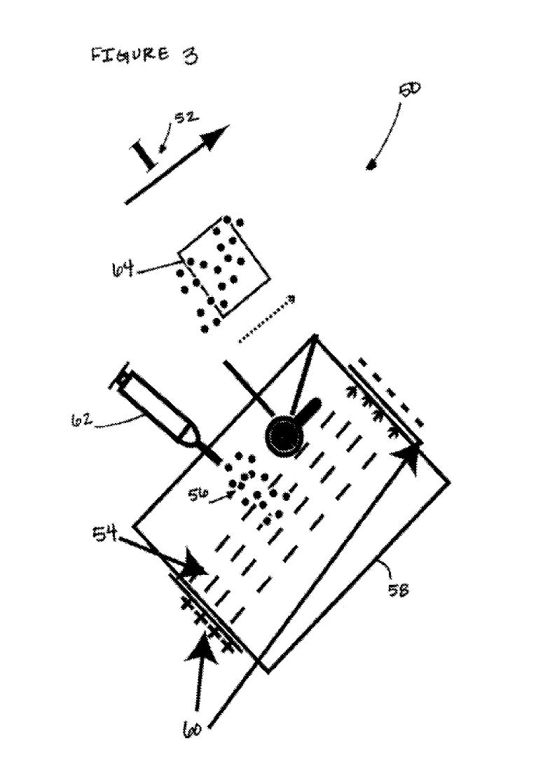

For example, FIG. 3 shows a set up 50 for generating an altered current with a newly generated displacement current 52 through the combined effects of an electric field 54 and a chemical agent 56. A tissue or composite of tissues 58 is placed within an electric source 60 which generates an electric field 54 and combined with chemical source 62 which releases a chemical agent 56 that can be focused on the tissue 58. In the area that the chemical agent 56 is released in the tissue 64, the electric field 54 transects the sub region of tissue 64, and the chemical agent 56 reacts with the sub region of tissue 64 to alter the tissue's relative permittivity relative to the applied electric field 54. This generates a displacement current 52 in addition to the current that would be present due to the source electric field 54. The chemical agent 56 may be any agent which can react with the tissue or cellular components of the tissue 64 to alter its permittivity relative to the electric field 54. This may be by a thermo reactive process to raise or lower the tissue 64 temperature or through a chemical reaction which alters the distribution of ions in the cellular and extra-cellular media, for instance, along ionic double layers at cell walls in the tissue 64. Similarly, the conformation of proteins and other charged components within the tissue 64 could be altered such that the permittivity of the tissue is altered relative to the low frequency electric field 54. The agent could also be any agent that adapts the permanent dipole moments of any molecules or compounds in the tissue 64, temporarily or permanently relative to the low frequency electric field 54. The chemical reaction driven by the chemical agent 56 must work rapidly enough such that the permittivity of the tissue is quickly altered in the presence of the electric field 54 in order to generate the displacement current 52. The reaction may also be such as to fluctuate the permittivity, such that as the permittivity continues to change displacement currents continue to be generated. In addition to the main permittivity change that occurs in the tissues, a conductivity change could also occur in the tissue, which secondarily alters the ohmic component of the currents. A biological agent may be used in place of, or in addition to, the chemical agent 56. This embodiment may have particular application for focused drug delivery where an additional chemical or biological agent is included to assist in therapy of the tissue, or where the altered current could drive an additional electrochemical reaction for therapy. For example, this could be used in areas such as focused gene therapy or focused chemotherapy.

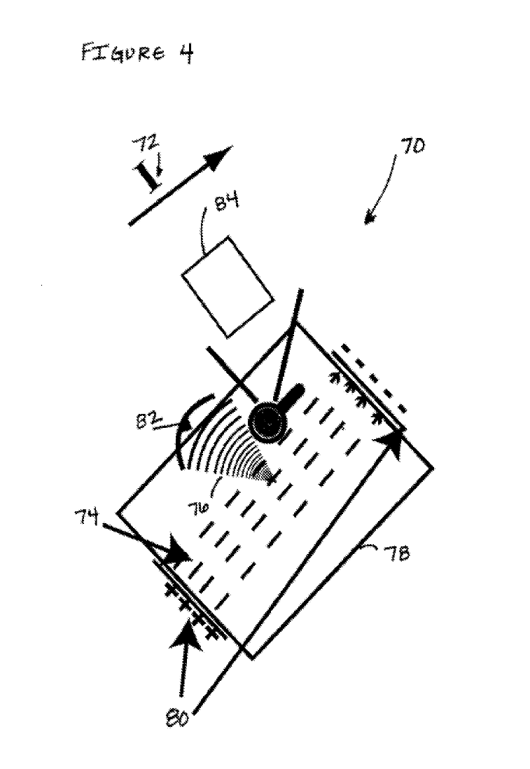

Another example is shown in FIG. 4, which illustrates a set up 70 for applying a method for generating an altered current with a newly generated displacement current 72 through the combined effects of a low frequency electric field 74 and an electromagnetic radiation field 76. A tissue or composite of tissues 78 is placed within a low frequency electric field 74 which is generated by an electric source 80 and combined with radiation source 82 which generates a radiation field 76 that can be focused on the tissue 78. In the area that the radiation field 76 is focused in the tissue 78, the electric field 74 transects the sub component of tissue 84, where the radiation field 76 interacts with the sub component of tissue 84 to alter the tissue's relative permittivity relative to the applied electric field 74, and as such generates a displacement current 72 in addition to the current that would be present due to the source electric field 74 or the radiation source field 76 alone. The electromagnetic radiation field 76 could, for example, interact with the tissue 84 by altering its temperature through ohmic processes, alter the distribution of ions in the cellular and extra-cellular media for instance along ionic double layers along cell walls through the electric forces acting on the ions, or alter the conformation of proteins and other charged components within the tissue through the electric forces such that the permittivity of the tissue is altered relative to the low frequency electric field 74. Furthermore, the electromagnetic field 76, could interact with the tissue 84 by moving components of the tissue via electrorestrictive forces, as would be seen in anisotropic tissues, to alter the continuum permittivity of the tissue relative to the low frequency electric field 74. In addition to the main permittivity change that occurs in the tissues, a conductivity change could also occur in the tissue, which secondarily alters the ohmic component of the currents.

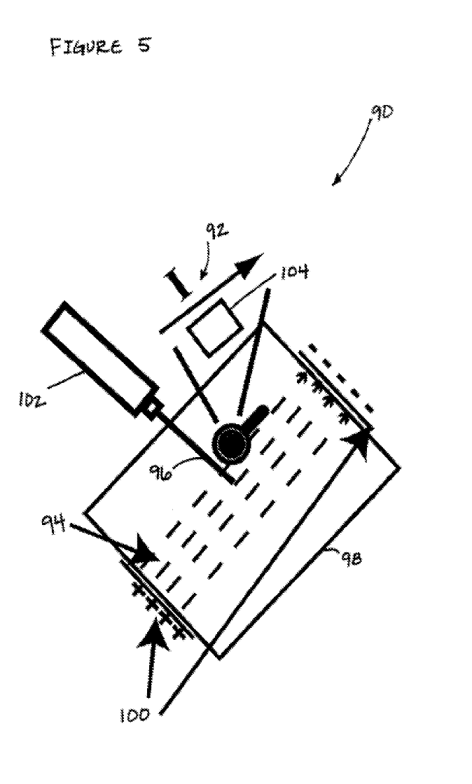

FIG. 5 shows a set up 90 for applying a method for generating an altered current with a newly generated displacement current 92 through the combined effects of an electric field 94 and an optical beam 96. A tissue or composite of tissues 98 is placed within electric field 94 generated by an electric source 100 and combined with optical source 102 which generates optical beam 96 that can be focused on the tissue 98. In the area that the optical beam 96 is focused on the tissue, the electric field 94 transects the sub component of tissue 104, where the optical beam 96 reacts with the tissue to alter the tissue's relative permittivity relative to the applied electric field 94, and as such generates a displacement current 92 in addition to the current that would be present due to the source electric field 94. The optical beam 96 could, for example, interact with the tissue by altering its temperature through photothermal effects and/or particle excitation, alter the distribution of ions in the cellular and extra-cellular media for instance along ionic double layers along cell walls by exciting the movement of ions optically, ionizing the tissue via laser tissue-interactions, or alter the conformation of proteins and other charged components within the tissue such that the permittivity of the tissue is altered relative to the low frequency electric field 94. In addition to the main permittivity change that occurs in the tissues, a conductivity change could also occur in the tissue, which secondarily alters the ohmic component of the currents.

In another embodiment, a thermal source to alter the permittivity of the tissue may be used. In such embodiments, a thermal source such as a heating probe, a cooling probe, or a hybrid probe may be placed external or internal to the tissue to be stimulated. A thermal source may alter the permittivity of the tissue through the direct permittivity dependence of tissue temperature, mechanical expansion of tissues in response to temperature changes, or by mechanical forces that arise due to altered particle and ionic agitation in response to the temperature alteration such that permittivity of the tissue is altered relative to an applied electric field. In addition to the main permittivity change that occurs in the tissues, a conductivity change could also occur in the tissue, which secondarily alters the ohmic component of the currents. This embodiment may be useful for stimulation in the presence of an acute injury to the tissue where the thermal source could be used to additionally assist in the treatment of the tissue injury, for example with a traumatic brain injury or an infarct in any organ such as the heart. The tissue could be cooled or heated at the same time stimulation is provided to reduce the impact of an injury.

In a further embodiment, the method according to the present disclosure is applied in the area of muscular stimulation, where amplified, focused, direction altered, and/or attenuated currents could be used to alter muscular activity via direct stimulation, depolarizing muscular cells, hyperpolarizing muscular cells, modifying membrane potentials, and/or increasing or decreasing the excitability of the muscle cells. This alteration of excitability or firing patterns can last past the duration of stimulation and thus be used as a basis to provide lasting treatment. Additionally, the stimulation can be provided in multiple, but separate sessions to have a summed, or carry-over effect, on the excitability of cells and tissue. Additionally, the stimulation could be provided to prime the tissue by adjusting the muscle cell excitability to make it more or less susceptible to alternate follow up forms of stimulation. The stimulation could be used after another form of stimulation was used to prime the tissue. Furthermore, the stimulation could be applied for a chronic period of time. This embodiment may be useful for altering or assisting cardiac pacing or function, assisted breathing, muscle stimulation for rehabilitation, muscle stimulation in the presence of nerve or spinal cord injury to prevent atrophy or assist in movement, or as substitution for physical exercise.

In yet another embodiment, the method according to the present disclosure can be applied the area of physical therapy, where amplified, focused, direction altered, and/or attenuated currents could be used to stimulate blood flow, increase or alter neuromuscular response, limit inflammation, speed the break down of scar tissue, and speed rehabilitation by applying the focus of the current generation to the effected region in need of physical therapy. It is envisioned that the method according to the present disclosure may have a wide variety in the area of physical therapy including the treatment or rehabilitation of traumatic injuries, sports injuries, surgical rehabilitation, occupational therapy, and assisted rehabilitation following neural or muscular injury. For instance, following an injury to a joint or muscle, there is often increased inflammation and scar tissue in the region and decreased neural and muscular response. Typically, ultrasound is provided to the affected region to increase blood flow to the region and increase the metabolic re-absorption of the scar tissue while electrical stimulation is provided separately to the nerves and muscles; however, by providing them together, a person could receive the benefit of each individual effect, but additionally amplified stimulatory and metabolic effects through the altered currents. The other methods for generating altered currents discussed within could also be used to assist in physical therapy via the displacement currents that are generated.

Furthermore, the method according to the present disclosure may be applied to the area of cellular metabolism, where currents could be used to interact with electrically receptive cells or charged membranes to alter the tissue or cellular dynamics. It is envisioned that this embodiment could provide treatment for various diseases where electrically receptive cells respond to the newly generated displacement currents and altered current distribution.

Furthermore, the method according to the present disclosure may be applied to the area of gene therapy. Amplified, focused, direction altered, and/or attenuated currents could be used to interact with electrically receptive cells or receptors within the cell to influence protein transcription processes and alter the genetic content of the cells. The altered current densities in the tissue can interact with the tissue to stimulate this altered gene regulation. Additionally, the displacement currents generated by the method could further be used to assist in drug delivery and/or gene therapy through the altered current influence on the delivery of agents.

All of the methods and processes discussed in this document could be implemented with any form of stimulation, including but not limited to electromagnetic, acoustic, optical, thermal, electrical, magnetic, and/or combined methods (and/or methods which alter tissue impedances relative to electrical sources to generate altered stimulation currents, for example with electromagnetic, chemical, acoustic, optical, thermal, electrical, magnetic, and/or combined sources). Typically, the two energy components would be simultaneously applied, but using one energy type to prime the tissue for one or more energy types is also possible, or sequentially applying the energies.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A method for modifying neural transmission patterns, the method comprising:
noninvasively applying a first type of energy to a region of neural tissue;
noninvasively applying a second type of energy to the region of tissue, wherein the combined effect modifies a neural transmission pattern between cells of the neural tissue.

2. The method according to claim 1, wherein the modification is selected from the group consisting of: establishing a transmission patterns between the cells, strengthening a transmission patterns between the cells, weakening a transmission patterns between the cells, removing a transmission patterns between the cells, and a combination thereof.

3. The method according to claim 1, wherein modifying transmission patterns results in long-term potentiation between the cells.

4. The method according to claim 1, wherein modifying transmission patterns results in long-term depression between the cells.

5. The method according to claim 1, wherein the neural tissue is tissue of a structure or multiple structures within the brain or the nervous system selected from the group consisting of: dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, and sub-cortical structures.

6. The method according to claim 1, wherein the affect of the modification alters neural function past the duration of stimulation.

7. The method according to claim 1, wherein the first type of energy is selected from the group consisting of: thermal, optical, mechanical, electromagnetic, and electrical.

8. The method according to claim 1, wherein the second type of energy is selected from the group consisting of: thermal, optical, mechanical, electromagnetic, and electrical.

9. The method according to claim 1, wherein the first type of energy is an electric field.

10. The method according to claim 9, wherein the electric field is pulsed.

11. The method according to claim 9, wherein the electric field is time varying.

12. The method according to claim 9, wherein the electric field is pulsed a plurality of time, and each pulse may be for a different length of time.

13. The method according to claim 9, wherein the electric field is time invariant.

14. The method according to claim 1, wherein the second type of energy is a mechanical field.

15. The method according to claim 14, wherein the mechanical field is generated by an ultrasound device.

16. The method according to claim 14, wherein the mechanical field is pulsed.

17. The method according to claim 14, wherein the mechanical field is time varying.

18. The method according to claim 14, wherein the mechanical field is pulsed a plurality of time, and each pulse may be for a different length of time.

19. A method for modifying neural transmission patterns, the method comprising:
noninvasively applying an electric field to a region of neural tissue; and
noninvasively applying a mechanical field to the region of neural tissue, wherein the combined effect modifies a neural transmission pattern between cells of the neural tissue.

20. The method according to claim 19, wherein the modification is selected from the group consisting of: establishing a transmission patterns between the cells, strengthening a transmission patterns between the cells, weakening a transmission patterns between the cells, removing a transmission patterns between the cells, and a combination thereof.

21. The method according to claim 19, wherein modifying transmission patterns results in long-term potentiation between the cells.

22. The method according to claim 19, wherein modifying transmission patterns results in long-term depression between the cells.

23. The method according to claim 19, wherein the neural tissue is tissue of a structure or multiple structures within the brain or the nervous system selected from the group consisting of: dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, and sub-cortical structures.

24. The method according to claim 19, wherein the affect of the modification alters neural function past the duration of stimulation.

25. A method for modifying neural transmission patterns, the method comprising:
providing a noninvasive transcranial neural stimulator; and
using the stimulator to modify a neural transmission pattern between cells of neural tissue.

26. The method according to claim 25, wherein the modification is selected from the group consisting of: establishing a transmission patterns between the cells, strengthening a transmission patterns between the cells, weakening a transmission patterns between the cells, removing a transmission patterns between the cells, and a combination thereof.

27. The method according to claim 25, wherein modifying transmission patterns results in long-term potentiation between the cells.

28. The method according to claim 25, wherein modifying transmission patterns results in long-term depression between the cells.

29. The method according to claim 25, wherein the neural tissue is tissue of a structure or multiple structures within the brain or the nervous system selected from the group consisting of: dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord.

30. The method according to claim 25, wherein the affect of the modification alters neural function past the duration of stimulation.
